SEQUENCE LISTING (14 sequences)

SEQ ID NO: 1 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 121 aa, synthetic construct
  VARIANT 1..121: note = IL-15 variant
NWVNVISDLKKIEDLIQSMHIDATLYTESDVHPSCKVTAMKCFLLELQVISLESGDASIHDTVENLIILANNSLSSNANVTESGCKECEELEEKNIKEFLQSFVHIVQMFINTSHPLTWKK

SEQ ID NO: 4 - protein, 122 aa, synthetic construct
  VARIANT 1..122: note = IL-15variant
NWVNVISDLKKIEDLIQSMHIDATLYTESDVHPSCKVTAMKCFLLELQVISLESGDASIHDTVENLIILANNSLSSNANVTESGCKECEELEEKNIKEFLQSFVHIVQMFINTSLRLISGRH

SEQ ID NO: 5 - protein, 123 aa, synthetic construct
  VARIANT 1..123: note = IL-15variant
NWVNVISDLKKIEDLIQSMHIDATLYTESDVHPSCKVTAMKCFLLELQVISLESGDASIHDTVENLIILANNSLSSNANVTESGCKECEELEEKNIKEFLQSFVHIVQMFINTSHSQLETGKK

SEQ ID NO: 6 - protein, 121 aa, synthetic construct
  VARIANT 1..121: note = IL-15variant
NWVNVISDLKKIEDLIQSMHIDATLYTESDVHPSCKVTAMKCFLLELQVISLESGDASIHDTVENLIILANNSLSSNANVTESGCKECEELEEKNIKEFLQSFVHIVQMFINTSLIERHHK

SEQ ID NO: 7 - protein, 122 aa, synthetic construct
  VARIANT 1..122: note = IL-15variant
NWVNVISDLKKIEDLIQSMHIDATLYTESDVHPSCKVTAMKCFLLELQVISLESGDASIHDTVENLIILANNSLSSNANVTESGCKECEELEEKNIKEFLQSFVHIVQMFINTSHVESGKKK

SEQ ID NO: 8 - protein, 6 aa, synthetic construct
  UNSURE 1..6: note = random sequence
LRLISG

SEQ ID NO: 9 - protein, 4 aa, synthetic construct
  UNSURE 1..4: note = random sequence
LIER

SEQ ID NO: 10 - protein, 5 aa, synthetic construct
  UNSURE 1..5: note = random sequence
HVESG

SEQ ID NO: 11 - protein, 7 aa, synthetic construct
  UNSURE 1..7: note = random sequence
HSQLETG

SEQ ID NO: 12 - protein, 5 aa, synthetic construct
  REGION 1..5: note = linker
GGGGS

SEQ ID NO: 13 - protein, 5 aa, synthetic construct
  UNSURE 1..5: note = random sequence
HPLTW

SEQ ID NO: 14 - protein, 4 aa, synthetic construct
  UNSURE 1..4: note = X3 peptide
RHHK

(19) United States
(12) Patent Application Publication
(10) Pub. No.: US 2025/0002552 A1
(43) Pub. Date: Jan. 2, 2025

(54) RECOMBINANT INTERLEUKIN-15 VARIANT

(71) Applicant: DRACOTERPIN BIOMEDICINE LLC, Sacramento, CA (US)

(72) Inventors: Yongtao LU, Shaanxi (CN); Jianguang LIU, Shaanxi (CN)

(73) Assignee: XI'AN LONGTENGJINGYUN BIOTECHNOLOGY CO., LTD., Shaanxi (CN)

(21) Appl. No.: 18/704,384

(22) PCT Filed: Aug. 11, 2022
(86) PCT No.: PCT/CN2022/111669

§ 371 Date: Apr. 24, 2024

(30) Foreign Application Priority Data
Oct. 26, 2021 (CN) 202111246297.6

(51) Int. Cl. C07K 14/54; A61K 38/00; C12N 15/70

(52) U.S. Cl. CPC C07K 14/5443; C12N 15/70; A61K 38/00

(57) ABSTRACT

Provided is a recombinant interleukin-15 variant, wherein one or more amino acid residues are added, deleted and substituted at the end of an amino acid sequence of a wild type interleukin-15. Thus, the carboxyl terminal of interleukin-15 forms an amphipathic alpha helix structure, and the carboxyl terminal of the interleukin-15 variant has a more stable structure than the wild type interleukin-15, so that the interleukin-15 variant can be highly expressed in Escherichia coli, and is easier to be separated and purified. The expression quantity of the interleukin-15 variant is 10-20 times that of the wild type interleukin-15.

The present application claims the priority of Chinese patent application No. 2021112462976 filed on Oct. 26, 2021.

### FIELD OF THE INVENTION

The present disclosure belongs to the field of molecular biology and specifically relates to a recombinant interleukin-15 variant.

### BACKGROUND OF THE INVENTION

Interleukin-15 (IL-15) is a cytokine of about 12-14 kD. It belongs to the same 4 a-helix cytokine family as IL-2, IL-4, IL-7, IL-9, granulocyte colony-stimulating factor, and granulocyte-macrophage colony-stimulating factor.

The native mature human IL-15 mature peptide contains 114 amino acids, including 4 cysteine residues (Cys35 and Cys85; Cys42 and Cys88), and the two pairs of intramolecular disulfide bonds formed between the cysteine residues play an important role in maintaining the spatial conformation and biological activity of IL-15.

The IL-15 receptor consists of 3 subunits (α, β and γ subunits). The α subunit of the IL-15 receptor has a high affinity for IL-15. IL-15 forms a complex with the α subunit (IL-15-Rα) in physiological state, enhancing the affinity of IL-15 for the β and γ chain subunits of the receptor and activating T cells and NK cells. Therefore, some pharmaceutical companies have complexed IL-15 with the α subunit (or a part thereof) of the IL-15 receptor, which has shown good biopotency and stability in animal tests.

IL-15 plays a role in the body's normal immune response, such as promoting the development of T cells, B cells and natural killer (NK) cells. Inflammation is closely related to the occurrence of tumors. As IL-15 can promote the proliferation of NK cells, B lymphocytes and T lymphocytes and maintain the function of these immune cells, IL-15 has certain efficacy in the treatment of some malignant tumors. In a variety of experimental animal tumor models (lung adenocarcinoma, melanoma, colon cancer, liver cancer, lymphoma), the treatment using IL-15 can promote tumor regression, reduce tumor metastasis, and improve the survival rate.

IL-15 has been found to be an effective vaccine adjuvant in clinical studies of infectious disease models. Steel et al. developed a DC vaccine (which can constitutively express IL-15) for the targeted treatment of neu+ breast cancer. Researchers evaluated IL-15 as an immune adjuvant to stimulate the activity of immune cells in vivo. The existing clinical results show that the NK cells, CD8+ T cells, CD8+ memory T cells and dendritic cells, cultured in medium supplemented with IL-15, have significantly improved biological activity after being transferred into animals.

With the deepening of research on the mechanism of action and clinical application of IL-15, the demand for IL-15 as a drug and an immune adjuvant has also increased greatly. However, there are obvious bottlenecks in the drug development of native wild type IL-15, including low expression amount in prokaryotes and eukaryotes, difficulty in purification, and short half-life. Commercially, prokaryotic expression systems are mainly used for the expression of IL-15, and the expression products of E. coli are usually in the form of inclusion bodies, which require complex denaturation-renaturation operations to obtain the active form of IL-15, which greatly limits the yield of active proteins. Multiple purification steps are often required to obtain proteins with high purity, resulting in reduced protein yields and reduced biological activity. Therefore, it is necessary to provide a more stable IL-15 in the field.

### SUMMARY OF THE INVENTION

### IL-15 Variants

The single-letter codes of amino acids used hereinafter are well known in the art, as described in JBC 243, p3558, 1968.

In one aspect, the present disclosure provides an IL-15 variant; when compared with the wild type human IL-15, the IL-15 variant comprises a polypeptide added at the carboxyl terminal of wild type human IL-15, wherein the polypeptide consists of 1 to 30 amino acid residue(s).

In some embodiments, without being bound by specific theories, the addition of a polypeptide with a length of no more than 30 amino acids to the carboxyl terminal of wild type human IL-15 facilitates the formation of amphipathic alpha helixes, and increases the expression in hosts (in especially prokaryotic hosts) by improving the stability of the carboxyl terminal structure. In one example, the expression amount is about 10 to 20 times that of wild type IL-15, or even higher.

In some embodiments, provided is an IL-15 variant with at least one basic hydrophilic amino acid residue added to the carboxyl terminal of wild type human IL-15, when compared to the wild type human IL-15.

In some embodiments, the basic hydrophilic amino acid is selected from the group consisting of H, R and K.

According to some embodiments, provided is an interleukin-15 variant comprising a peptide structure as shown in Formula (I) or Formula (II), from left to right in the direction of the amino terminal to the carboxyl terminal:

$${X}_{1} - J - {X}_{2} - {X}_{3} \qquad Formula ( I )$$

$${X}_{1} - {X}_{2} - J - {X}_{3} \qquad Formula ( II )$$

wherein:
X₁ represents a wild type human IL-15;
- represents a covalent bond, preferably an amide bond;
X₂ represents a random sequence or is absent, and the length of the random sequence is 3 to 8 amino acids in length, preferably 4 to 8 amino acids in length; the random sequence does not comprise an hydrophilic peptide residue;
X₃ represents a basic hydrophilic peptide residue selected from the group consisting of the following or a combination thereof: KK, HH, RH, KKK, HHK, RHHK (SEQ ID NO: 14);
J represents a linker or is absent.

The order of the linker and the random sequence is interchangeable without affecting the improved performance of the IL-15 variant.

The term “linker” is used in the present disclosure to link the wild type IL-15 to the added peptide, so as to ensure proper folding and stability of the protein. If the sequence of the linker is too short, it may affect the folding of the peptide chain, which would then interfere with each other; if the sequence of the linker is too long, it may involve immunogenicity problems, etc.

In some embodiments, the linker comprises at least one amino acid residue selected from the goup consisting of G and S.

In some embodiments, the linker is selected from the group consisting of the following or a combination thereof: (G)ₙ, (GS)ₙ, (GGGGS)ₙ (SEQ ID NO: 12) and (GGS)ₙ, wherein n is an integer from 1 to 10 (1, 2, 3, 4, 5, 6, 7, 8, 9, 10; preferably 1, 2, 3, 4, 5, 6, 7).

In some particular embodiments, provided is an IL-15 variant comprising a structure selected from the group consisting of the following (or as shown in the following structures):
X₁-HPLTW (SEQ ID NO: 13)-KK,
X₁-LRLISG (SEQ ID NO: 8)-RH,
X₁-LIER (SEQ ID NO: 9)-HHK,
X₁-LIE-RHHK,
X₁-HVESG (SEQ ID NO: 10)-KKK, and
X₁-HSQLETG (SEQ ID NO: 11)-KK;
wherein, X₁ represents wild type human IL-15.

In the present disclosure, a variant refers to a polypeptide produced after the introduction of substitution, addition, deletion or modification of amino acid residues to the parent polypeptide. The variant has improved physical, chemical or biological activity compared to the activity of the parent polypeptide.

In some embodiments, the wild type human IL-15 is selected from the group consisting of a naturally occurring human IL-15 and a naturally occurring human IL-15 splice variant.

In some particular embodiments, the wild type human IL-15 is a mature form of a wild type human IL-15. In some particular embodiments, the wild type human IL-15 is as shown in SEQ ID NO: 2.

In the present disclosure, the terms “interleukin-15” and “IL-15” refer to interleukin-15 protein or polypeptide, especially human interleukin-15 protein or polypeptide. The immature form of IL-15 comprises 162 amino acids (SEQ ID NO: 1), wherein amino acids at positions 1-29 constitute a signal peptide and amino acids at positions 30-48 constitute a propeptide; the mature form of IL-15 corresponds to amino acids at positions 49 to 162. The immature form of IL-15 is available at UniProtKB Accession No. P40933. The amino acid sequence of human mature IL-15 corresponds to SEQ ID NO: 2. The term “IL-15” also includes any variant or isoform of IL-15 that is naturally expressed by cells. Notably, several spliced transcript variants of IL-15 have been reported.

In the present disclosure, “polypeptide” is understood as a linear or cyclic polymer formed by the linkage of at least two amino acids via peptide bonds. In some cases, a polypeptide comprises the 20 types of naturally occurring amino acids; in other cases, a polypeptide may comprise amino acids in addition to the 20 types of amino acids encoded by the genetic code; and in still other cases, a polypeptide may comprise modified amino acids (e.g., post-translation modifications, or through a chemical process). These modifications can occur at any position of the polypeptide, in the peptide backbone, in the side chain, or at the carboxyl or amino terminal, for example, but not limited to, acylation, ribosylation, disulfide bond formation, demethylation, formylation, methylation, myristoylation, oxidation, hydroxylation, phosphorylation, glycosylation, pegylation, GPI anchor formation, sulfation, ubiquitination, covalent or non-covalent cross-linking, cyclization, and immobilization to lipids or lipid derivatives.

In some embodiments, the IL-15 variant of the present disclosure comprises a polypeptide selected from the group consisting of SEQ ID NO: 3, SEQ ID NO: 4, SEQ ID NO: 5, SEQ ID NO: 6 and SEQ ID NO:7.

In some embodiments, the IL-15 variant of the present disclosure has at least 85%, 86%, 87%, 88%, 89%, 90%, 91%, 92%, 93%, 94%, 95%, 96%, 97%, 98%, 99% identity with any of SEQ ID NOs: 3 to 7.

### Polynucleotides

According to some embodiments, the present disclosure provides a polynucleotide encoding the IL-15 variant of the present disclosure.

The term “polynucleotide” or “nucleic acid molecule” refers to a polymer comprising nucleotides. Examples of polynucleotides are, for example, but not limited to, DNA, RNA, LNA and cDNA.

Those skilled in the art understand that the nucleotide sequences encoding the same amino acid sequence can be different. Due to codon degeneracy and codon bias in different hosts, different nucleotide sequences can encode the same amino acid sequence, and these sequences all fall within the scope of the present disclosure.

### Expression Vectors

According to some embodiments, the present disclosure provides a recombinant expression vector comprising the polynucleotide of the present disclosure.

The expression vector can be a prokaryotic or eukaryotic expression vector, or a shuttle vector.

Expression vectors are well-known in the art and are usually commercially available. When used to express the active molecule of the present disclosure, those skilled in the art insert the coding sequence into an appropriate site of the expression vector.

Examples of expression vectors are for example, but not limited to, PET expression vectors, for example pET Dsb 39b, pET Expression System 33b, pET GST Fusion Systems 41, and pET NusA Fusion Systems 43.1; PGEX expression vectors, for example pGEX-2T, pGEX-2TK, pGEX-3X, pGEX-4T-1, pGEX-4T-2, pGEX-4T-3, pGEX-5X-1, pGEX-5X-2, pGEX-5X-3,pGEX-6P-1, pGEX-6P-2, and pGEX-6P-3; PTYB expression vectors, for example PTYB1, PTYB2, PTYB11, and PTYB12; eukaryotic expression vectors, for example pCDNA3.1 (−), pCDNA3.1 (+), pPICZ alpha A, pGAPZαA, PYES2.0, pBI121, pEGFP-N1, pEGFP-C1, pPIC9K, and pPIC3.5K.

There are no particular limitations on the expression vector in the present disclosure. Those skilled in the art have the ability to select appropriate expression vectors according to the subsequent application, host type, production scale and other factors based on general knowledge.

### Host cells

The present disclosure provides a host cell comprising an expression vector according to the present disclosure, or expressing an IL-15 variant according to the present disclosure.

In some embodiments, the host cell comprises (for example it has been transformed or transfected with) an expression vector comprising the polynucleotide according to the present disclosure.

In some embodiments, the host cell is a prokaryotic cell.

In some embodiments, the host cell is an eukaryotic cell.

In some embodiments, the host cell is a bacterial, yeast or mammalian cell, in particular Pichia pastoris or Saccharomyces cerevisiae .

In some particular embodiments, the host cell is a prokaryotic microorganism, such as E. coli .

In some particular embodiments, the host cell is an eukaryotic cell.

In some embodiments, host cells such as plant and insect cells that express glycosylated peptides are used. Vertebrate cells can also be used as the host cell, for example, mammalian cell lines in suspension culture, monkey kidney CV1 cell line (COS-7), human embryonic kidney cell line (293 or 293T cells), baby hamster kidney cells (BHK), mouse sertoli cells (TM4 cells), monkey kidney cells (CV1), VERO-76, human cervical cancer cells (HELA), canine kidney cells (MDCK), buffalo rat liver cells (BRL3A), human lung cells (W138), human liver cells (Hep G2), mouse breast tumor cells (MMT060562), MRC5 cells, FS4 cells, CHO cells, myeloma cell lines (such as YO, NS0, P3X63 and Sp2/0).

In the present disclosure, the host cell cannot develop into an individual animal or an individual plant.

### Production Methods

The present disclosure provides a method for producing an IL-15 variant.

In some embodiments, a polynucleotide encoding an IL-15 variant is linked to an expression vector and transferred into an expression host to induce the expression of the IL-15 variant.

In some embodiments, the IL-15 variant is expressed as an inclusion body, which is subjected to renaturation.

In some embodiments, the inclusion body is dissolved in a urea solution (e.g., 8 M) and the IL-15 variant is purified by ion exchange and/or reversed-phase chromatography.

In some particular embodiments, the expression vector is prokaryotic pET41a, and the expression host is E. coli BL21 (DE3) or C41 (DE3).

### Pharmaceutical Compositions

The present disclosure provides a pharmaceutical composition comprising the IL-15 variant according to the present disclosure, and optionally further comprising a pharmaceutically acceptable diluent, carrier or excipient.

The term “pharmatically acceptable” means not the absence of undesirable in vivo or in vitro effect.

The term “a pharmaceutically acceptable diluent, carrier or excipient” refers to any component present in a pharmaceutical preparation (or a pharmaceutical composition) other than the active ingredient. Therefore, mention may be made of diluent, adhesive, lubricant, disintegrant, filler, colorant, wetting agent, emulsifier, pH buffer, preservative, etc. Pharmaceutically acceptable diluents, carriers or excipients are described in detail in the textbook Remington's The Science and Practice of Pharmacy.

Exemplary compositions may comprise one or more pharmaceutically acceptable components, for example stabilizer, antimicrobial agent, buffer, colorant, flavoring, adjuvant, etc.

The active molecule of the present disclosure (the IL-15 variant) together with a conventionally used carrier (or diluent, or excipient) is formulated into the form of a pharmaceutical composition and an unit dose thereof.

The composition may be in solid form, for example a tablet or a filled capsule, or in freeze-dried form. When the composition of the present disclosure is a solid preparation, it is reconstituted with a suitable medium before use.

The composition can be in liquid form, for example injection, solution, suspension and emulsion. Exemplary compositions are used for oral administration, or for parenteral (including subcutaneous) administration as a sterile injectable solution. When the composition of the present disclosure is a liquid preparation, it includes, but is not limited to, aqueous or oily suspensions, solutions, emulsions and syrups. Liquid forms suitable for oral administration may include aqueous or non-aqueous media containing buffers, suspensions and dispersants, colorants, flavorings, etc. Suspensions include, but are not limited to, sorbitol syrup, methylcellulose, glucose, gelatin, hydroxyethylcellulose, carboxymethylcellulose and aluminum stearate gel. Emulsifiers include, but are not limited to, lecithin, sorbitan monooleate and gum arabic. Non-aqueous media include, but are not limited to, edible oil, propylene glycol and ethanol. Preservatives include, but are not limited to, methylparaben, propylparaben and sorbic acid.

In some particular embodiments, the pharmaceutical composition may be a lyophilized preparation or an injectable solution.

In some particular embodiments, the injectable composition is usually based on injectable sterile saline or phosphate-buffered saline.

In some particular embodiments, a unit dose of the pharmaceutical composition may comprise 1% to 10% of the IL-15 variant by mass, in particular 1%±10% thereof, 2%±10% thereof, 3%±10% thereof, 4%±10% thereof, 5%±10% thereof, 6%±10% thereof, 7%±10% thereof, 8%±10% thereof, 9%±10% thereof, 10%±10% thereof.

In some particular embodiments, a unit dose of the pharmaceutical composition comprises the IL-15 variant of 0.25 μg/kg to 100 μg/kg, for example, but not limited to, 1 μg/kg, 5 μg/kg, 10 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg±10% thereof.

In some embodiments, the IL-15 variant or pharmaceutical composition of the present disclosure is administered in multiple doses. In some embodiments, the dose is 0.25 μg/kg for the first cycle (28 days/cycle), followed by 0.5 μg/kg, 1 μg/kg, 2 μg/kg, 4 μg/kg, and 8 μg/kg for successive cycles.

In some embodiments, the dose is 1 μg/kg in the first cycle, 2 μg/kg in the second cycle, 4 μg/kg in the third cycle, and 8 μg/kg in the fourth cycle. For example, a subject may receive the same dose three times a week for two continuous weeks, and then have two-week intervals between each treatment cycle. In other embodiments, the IL-15 variant or pharmaceutical composition of the present disclosure may be administered subcutaneously (s.c.).

### Uses and Treatment Methods

The present disclosure provides an IL-15 variant for use in the prevention or treatment of an infectious disease. In some embodiments, the infectious disease is selected from the group consisting of smallpox virus infection, HIV infection, bacterial infection, fungal infection and HBV infection.

The present disclosure provides an IL-15 variant for use in the prevention or treatment of a cancer. In some embodiments, the cancer is selected from the group consisting of melanoma, colorectal cancer, skin cancer, lymphoma, renal cell carcinoma, liver cancer, lung cancer, gastric cancer and breast cancer.

The present disclosure provides an IL-15 variant for use in the prevention or treatment of a hematological disease. In some embodiments, the hematological disease is selected from the group consisting of anemia, leukemia and myelodysplastic syndrome.

The present disclosure provides an IL-15 variant for use in the prevention or treatment of an inflammatory disease. In some embodiments, the inflammatory disease is selected from the group consisting of autoimmune disease, celiac disease, sarcoidosis, ulcerative colitis, Crohn's disease, cholangitis, uveitis and dermatitis; in some embodiments, the autoimmune disease is selected from the group consisting of multiple sclerosis, psoriasis, rheumatoid arthritis, gastritis and mucositis.

The present disclosure provides use of the aforementioned IL-15 variant in the preparation of a medicament for the prevention or treatment of a disease selected from the group consisting of infectious disease, cancer, hematological disease and inflammatory disease. In some embodiments, the infectious disease is selected from the group consisting of smallpox virus infection, HIV infection, bacterial infection, fungal infection and HBV infection. In some embodiments, the cancer is selected from the group consisting of melanoma, colorectal cancer, skin cancer, lymphoma, renal cell carcinoma, liver cancer, lung cancer, gastric cancer and breast cancer. In some embodiments, the hematological disease is selected from the group consisting of anemia, leukemia and myelodysplastic syndrome. In some embodiments, the inflammatory disease is selected from the group consisting of autoimmune disease, celiac disease, sarcoidosis, ulcerative colitis, Crohn's disease, cholangitis, uveitis and dermatitis; in some embodiments, the autoimmune disease is selected from the group consisting of multiple sclerosis, psoriasis, rheumatoid arthritis, gastritis and mucositis.

The present disclosure provides a method for the prevention or treatment of the following diseases: infectious disease, cancer, hematological disease and inflammatory disease. The method includes a step of administering a prophylactically or therapeutically effective amount of the IL-15 variant of the present disclosure to a subject. In some embodiments, the infectious disease is selected from the group consisting of smallpox virus infection, HIV infection, bacterial infection, fungal infection and HBV infection. In some embodiments, the cancer is selected from the group consisting of melanoma, colorectal cancer, skin cancer, lymphoma, renal cell carcinoma, liver cancer, lung cancer, gastric cancer and breast cancer. In some embodiments, the hematological disease is selected from the group consisting of anemia, leukemia and myelodysplastic syndrome. In some embodiments, the inflammatory disease is selected from the group consisting of autoimmune disease, celiac disease, sarcoidosis, ulcerative colitis, Crohn's disease, cholangitis, uveitis and dermatitis; in some embodiments, the autoimmune disease is selected from the group consisting of multiple sclerosis, psoriasis, rheumatoid arthritis, gastritis and mucositis.

In some embodiments, the subject in need of treatment, such as a patient or an individual, is usually a mammal, for example a human.

In some embodiments, the IL-15 variant or the pharmaceutical composition of the present disclosure is administered to a subject at least twice a day, at least once a day, at least once every 48 hours, at least once every 72 hours, at least once a week, at least once every 2 weeks, at least once a month, at least once every 2 months, or at least once every 3 months.

In some embodiments, the IL-15 variant or the pharmaceutical composition of the present disclosure is administered via any route, for example, administered by parenteral injection (for example, subcutaneous or intravenous injection).

### DESCRIPTION OF THE DRAWINGS

FIG. 1: SDS-PAGE electropherogram of induced expression of wild type IL-15 in different E. coli single clones.

FIGS. 2A to 2E: SDS-PAGE electropherogram of expression of the IL-15 variant of the present disclosure in E. coli single clones. FIGS. 2A to 2E represent Variant 1 to Variant 5, respectively.

FIG. 3: HPLC quantitative results.

FIG. 4: Diagram of the three-dimensional structure of the IL-15 variant of the present disclosure. In the polar environment of an aqueous solution, the IL-15 variant can stably maintain intrachain hydrogen bonds and reduce the unwinding effect caused by water molecules.

### DETAILED DESCRIPTION OF THE INVENTION

The present disclosure will be further illustrated by the Examples below, and it should be understood that these Examples are only for illustrative purposes and are not intended to limit the protection scope of the present disclosure.

Experimental methods that do not specify the specific conditions in the Examples below are usually in accordance with conventional conditions, for example the conditions described in Sambrook et al., Molecular Cloning: A Laboratory Manual, Cold Spring Harbor Laboratory Press, 1989, or in accordance with the conditions recommended by the manufacturers. Unless otherwise specified, the reagents used are commercially available or publicly available reagents.

### EXAMPLES

### Example 1. Expression of Wild Type IL-15 and IL-15 Variants in E. coli

1. Construction of expression vectors

The nucleotide sequences of wild type IL-15 and IL-15 variants were synthesized by Tianjin KMD Bioscience Co., Ltd. The carboxyl terminal of wild type IL-15 was modified by introducing the sequence to be added into the reverse primer. When those skilled in the art know the amino acid sequence to be added, they can design and synthesize primers in accordance with well-known primer design principles using any available primer design software, which is within the competence of those skilled in the art.

The amino acid sequences of wild type IL-15 and constructed IL-15 variants are as shown in Table 1.

TABLE 1
Amino acid sequences of wild type IL-15 and IL-15 variants

| | | SEQ |
|---|---|---|
| | | ID |
| | Sequence | NO. |
| Wild type | Precursor sequence: | 1 |
| IL-15* | MRISKPHLRSISIQCYLCLLLNSHFLTEAGIHVFILGCFSAG | |
| | LPKTEANWVNVISDLKKIEDLIQSMHIDATLYTESDVHPSC | |
| | KVTAMKCFLLELQVISLESGDASIHDTVENLIILANNSLSS | |
| | NGNVTESGCKECEELEEKNIKEFLQSFVHIVQMFINTS | |
| | NWVNVISDLKKIEDLIQSMHIDATLYTESDVHPSCKVTAM | 2 |
| | KCFLLELQVISLESGDASIHDTVENLIILANNSLSSNGNVTE | |
| | SGCKECEELEEKNIKEFLQSFVHIVQMFINTS | |
| Variant 1 | NWVNVISDLKKIEDLIQSMHIDATLYTESDVHPSCKVTAM | 3 |
| | KCFLLELQVISLESGDASIHDTVENLIILANNSLSSNANVTE | |
| | SGCKECEELEEKNIKEFLQSFVHIVQMFINTSHPLTWKK | |
| Variant 2 | NWVNVISDLKKIEDLIQSMHIDATLYTESDVHPSCKVTAM | 4 |
| | KCFLLELQVISLESGDASIHDTVENLIILANNSLSSNANVTE | |
| | SGCKECEELEEKNIKEFLQSFVHIVQMFINTSLRLISGRH | |
| Variant 3 | NWVNVISDLKKIEDLIQSMHIDATLYTESDVHPSCKVTAM | 5 |
| | KCFLLELQVISLESGDASIHDTVENLIILANNSLSSNANVTE | |
| | SGCKECEELEEKNIKEFLQSFVHIVQMFINTSHSQLETGK | |
| | K | |
| Variant 4 | NWVNVISDLKKIEDLIQSMHIDATLYTESDVHPSCKVTAM | 6 |
| | KCFLLELQVISLESGDASIHDTVENLIILANNSLSSNANVTE | |
| | SGCKECEELEEKNIKEFLQSFVHIVQMFINTSLIERHHK | |
| Variant 5 | NWVNVISDLKKIEDLIQSMHIDATLYTESDVHPSCKVTAM | 7 |
| | KCFLLELQVISLESGDASIHDTVENLIILANNSLSSNANVTE | |
| | SGCKECEELEEKNIKEFLQSFVHIVQMFINTSHVESGKKK | |

Bold indicates the added peptide.
*The Accession Nos. of human IL-15 in databases include, for example but not limited to, UniProtKB database P40933, RefSeq No. NP_000576.1 and NP_751915.1.

2. PCR amplification was performed in accordance with conventional methods.
3. The expression vectors were enzymatically digested in a 30 μL reaction system, mixed well with the restriction enzyme and incubated at 37° C. overnight.
4. The PCR products and digested vectors were recovered.
5. The vector and the fragment were reacted at 37° C. at a molar ratio of 2:1 for 30 min, and were immediately cooled on ice and transformed into DH5α.
6. 0.5 mL of Amp-containing LB medium was added to a 1.5 mL tube and a single colony was inoculated into the tube for a total of 5 tubes, which were cultured under shaking in a shaker at 37° C. After 3 h of incubation, 1 μL was used as template to perform PCR. After PCR, the product was detected with agarose gel electrophoresis, and positive clones were selected for sequencing.
7. Storage of positive bacterial culture and extraction of plasmids: Positive clones sequenced as having the correct sequence were inoculated into 5 mL of Amp-containing LB liquid medium with an inoculation amount of 10 μL. 500 μL of bacterial culture was added to a 1.5 mL tube, 500 μL of 40% glycerol was added, and the tube was placed at −80° C. for storage. The bacterial culture was centrifuged and the bacteria were collect for plasmid extraction.
8. Expression of wild type IL-15 and IL-15 variants
a) 10 μl of plasmid was added to 100 μl of BL21 (DE3) competent cells, mixed immediately, and placed on ice for 30 min;
b) the mixture was heat shocked at 42° C. for 90 s, put into an ice bath rapidly for 5 min, spread on LB (containing 50 μg/ml of kanamycin) plates, cultured at 37° C. for 14 h, and then single clones were picked for induction culture.
9. Small-scale expression
a) Single clones were picked from the LB plates and activated by adding 2 ml of LB liquid medium (containing 50 μg/ml of kanamycin);
b) the bacterial culture cultured overnight was transferred into 20 ml of Amp-containing LB medium at a ratio of 1:50 and cultured at 37° C., 220 rpm until the OD₆₀₀ of the bacterial culture reached 0.6-0.8; the bacterial culture was transferred into 5 ml of LB liquid medium (containing 50 μg/ml of kanamycin) and cultured until the OD₆₀₀ of the bacterial culture reached 0.6-0.8; IPTG at a concentration of 1 mM was added to induce expression at 37° C. for 4 h.
10. SDS-PAGE identification of expression amount
a) OD600 of the culture medium was determined, 10 OD of bacterial culture was collected and centrifuged at 10,000 rpm for 2 min, and the supernatant was discarded;
b) the cells were resuspended with 1 mL of lysis buffer (10 mM Tris-HCl, pH 8.0) and placed on ice for ultrasonic disruption of cells under the ultrasonication conditions of 130 W, 4 min, on 3 s, off 3 s;
c) after completion of ultrasonication, 80 μl of sample was added to 20 μl of 5× loading buffer, heated at 95° C. for 5 min, and 12.5 μl (0.1 OD) of each sample was resolved on SDS-PAGE electrophoresis.

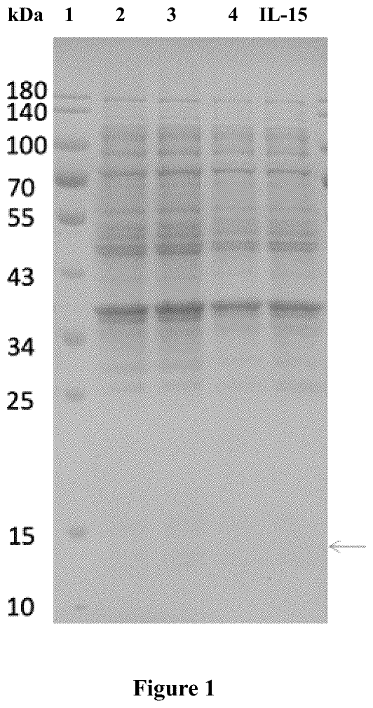

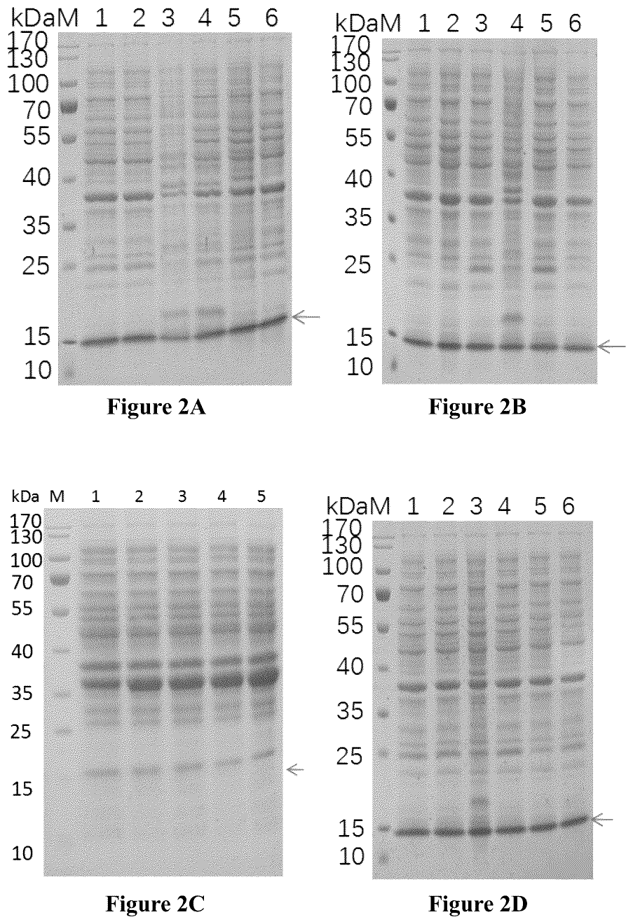

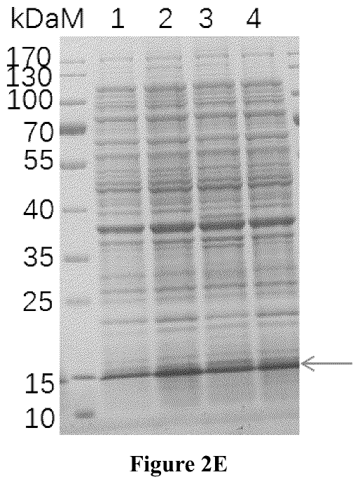

The expression of wild type IL-15 is as shown in FIG. 1. Expression of wild type IL-15 is not detectable in the lysate, supernatant or pellet. The numbers of 1, 2, 3 and 4 of FIG. 1 represent four different expression vectors. Compared to wild type interleukin-15, the expression levels of IL-15 Variant 1 to Variant 5 were statistically significantly improved in E. coli (p<0.05), with an increase of about 10 to 20 times (as shown in FIG. 2A to FIG. 2E).
11. HPLC identification of expression amount
a) 10 OD cells after expression were collected and resuspended with 1 ml buffer of 10 mM Tris-HCl, pH 8.0;
b) ultrasonic disruption was performed;
c) the disrupted sample was centrifuged at 12,000 rpm for 10 min, and the supernatant was discarded;
d) the pellet was dissolved with 1 ml of freshly prepared 8 M urea/10 mM Tris-HCl, pH 8.0, 10 mM DTT at room temperature under shaking for about 1 h;
e) the sample was filtered with a 0.2 μm filter and loaded for high-performance liquid chromatography analysis.

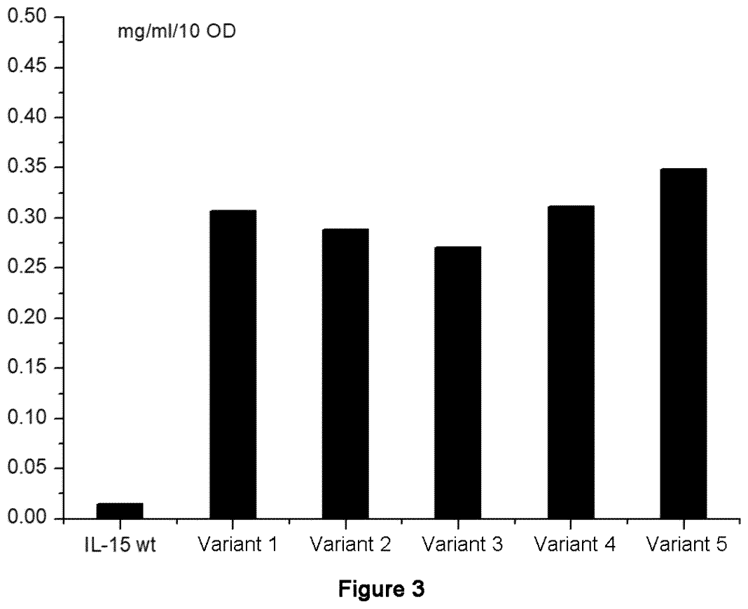

Analysis was performed using C4 columns, with 0.1% TFA in deionized water as mobile phase A, and 0.1% TFA in acetonitrile as mobile phase B, with a gradient from 20% B to 60% B for 15 minutes. The HPLC quantitative results are as shown in FIG. 3. The expression amounts of IL-15 Variant 1 to Variant 5 are approximately 20 times higher than that of wild type IL-15.

### Example 2. Molecular Dynamics of IL-15 Variants in Aqueous Solution

For IL-15 Variant 1 to Variant 5, homology modeling was performed using the SIWSS-model. The structure obtained by homology modeling was used as the initial structure, and molecular dynamics simulation for 200 ns was performed using the GROMACS software. OPLS-AA was used as the force field, and TIP4P was used as the water molecule model.

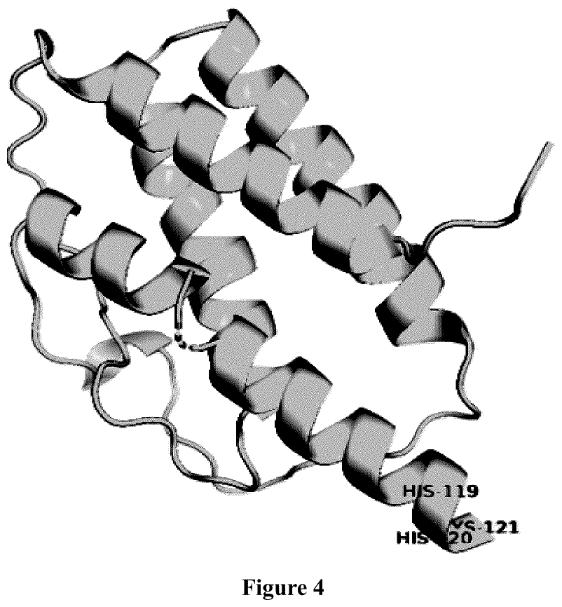

After a long range dynamic simulation of up to 200 ns, the average structure of the IL-15 variant was finally obtained. The protein structure was analyzed using the PyMOL software. It can be seen that the characteristic sequence added to the carboxyl terminal of the protein maintains the structure of alpha helix in the dynamic simulation under aqueous solution environment for prolonged time. This demonstrates that the added peptide designed in the present disclosure can stably maintain the intrachain hydrogen bond structure under polar environment of aqueous solution, so as to reduce the unwinding effect caused by water molecules (Variant 4 as shown in FIG. 4, and the results of other variants are similar to that of Variant 4).

1. An interleukin-15 variant, comprising 1 to 30 amino acid residue(s) added at the carboxyl terminal of a wild type human interleukin-15, the amino acid residue(s) comprising at least one basic hydrophilic amino acid residue selected from the group consisting of H, R and K;
the wild type human interleukin-15 is selected from the group consisting of a naturally occurring human interleukin-15, and a naturally occurring human interleukin-15 splice variant;
the wild type human interleukin-15 is a mature form of a wild type human interleukin-15.

2. The interleukin-15 variant according to claim 1, comprising a peptide structure as shown in Formula (I) or Formula (II), from left to right in the direction of the amino terminal to the carboxyl terminal:
$${X}_{1} - J - {X}_{2} - {X}_{3} \qquad Formula ( I )$$
$${X}_{1} - {X}_{2} - J - {X}_{3} \qquad Formula ( II )$$
wherein:
X₁ represents a wild type human interleukin-15;
- represents an amide bond;
J represents a linker or is absent;
X₂ represents a random sequence or is absent, and the length of the random sequence is 3 to 8 amino acids in length;
X₃ represents a basic hydrophilic peptide residue selected from the group consisting of the following or a combination thereof:
KK, HH, RH, KKK, HHK and RHHK;
the linker is selected from the group consisting of the following or a combination thereof: (G)ₙ, (GGGGS)ₙ, (GS)ₙ and (GGS)ₙ, where n is an integer from 1 to 10.

3. The interleukin-15 variant according to claim 2, comprising any of the following structures:
X₁-HPLTW-KK,
X₁-LIE-RHHK,
X₁-LIER-HHK,
X₁-HVESG-KKK,
X₁-LRLISG-RH, and
X₁-HSQLETG-KK.

4. The interleukin-15 variant according to claim 1, comprising a polypeptide as shown in the sequence selected from the group consisting of:
SEQ ID NO: 3, SEQ ID NO: 4, SEQ ID NO: 5, SEQ ID NO: 6, SEQ ID NO: 7; and a polypeptide having at least 85%, 86%, 87%, 88%, 89%, 90%, 91%, 92%, 93%, 94%, 95%, 96%, 97%, 98%, 99% identity with any of SEQ ID NOs: 3 to 7.

5. A polynucleotide encoding the interleukin-15 variant according to claim 1.

6. A recombinant expression vector comprising the polynucleotide according to claim 5.

7. A host cell,
comprising the recombinant expression vector according to claim 6; or
expressing the interleukin-15 variant according to claim 1;
the host cell cannot develop into an individual animal or an individual plant.

8. A pharmaceutical composition comprising:
the interleukin-15 variant according to claim 1, and
a pharmaceutically acceptable carrier or excipient or diluent.

9. A protein complex comprising the interleukin-15 variant according to claim 1.

10. A method for prevention or treatment of a disease selected from the group consisting of infectious disease, cancer, hematological disease and inflammatory disease, comprising step of administering a prophylactically effective amount of or therapeutically effective amount of the interleukin-15 variant according to claim 1 in a subject.

11. A method for increasing the expression amount of interleukin-15 in a host cell, comprising:
culturing the host cell according to claim 7 in a culture medium;
harvesting the interleukin-15 variant according to claim 1.

12. The interleukin-15 variant according to claim 1, the wild type human interleukin-15 is as shown in SEQ ID NO: 2.

13. The method of claim 10, wherein:
the infectious disease is selected from the group consisting of smallpox virus infection, HIV infection, bacterial infection, fungal infection and HBV infection.

14. The method of claim 10, wherein:
the cancer is selected from the group consisting of melanoma, colorectal cancer, skin cancer, lymphoma, renal cell carcinoma, liver cancer, lung cancer, gastric cancer and breast cancer.

15. The method of claim 10, wherein:
the hematological disease is selected from the group consisting of anemia, leukemia and myelodysplastic syndrome.

16. The method of claim 10, wherein:
the inflammatory disease is selected from the group consisting of autoimmune disease, celiac disease, sarcoidosis, ulcerative colitis, Crohn's disease, cholangitis, uveitis and dermatitis; the autoimmune disease is selected from the group consisting of multiple sclerosis, psoriasis, rheumatoid arthritis, gastritis and mucositis.

17. The method of claim 11, wherein:
the host cell is a prokaryotic host cell.
